(19) United States
(12) Patent Application Publication
(10) Pub. No.: US 2024/0312577 A1
(43) Pub. Date: Sep. 19, 2024

(54) SYSTEM AND METHOD FOR PROVIDING PERSONALIZED, PREVENTIVE AND CURATIVE CARE

(71) Applicants: Dharinder Kumar Tayal, Chandigarh (IN); Anushka Tayal, Chandigarh (IN); Asavari Tayal, Chandigarh (IN)

(72) Inventors: Dharinder Kumar Tayal, Chandigarh (IN); Anushka Tayal, Chandigarh (IN); Asavari Tayal, Chandigarh (IN)

(21) Appl. No.: 18/475,765

(22) Filed: Sep. 27, 2023

(30) Foreign Application Priority Data
Jul. 30, 2022 (IN) 202211043705

(51) Int. Cl. G16H 10/60

(52) U.S. Cl. CPC G16H 10/60

(57) ABSTRACT

Disclosed herein is a novel system and method for providing personalized predictive, preventive, palliative, and curative care. A system for determining suggestions to a user, comprising, (a) one or more user devices comprising one or more sensors, an input unit, an output unit and a transceiver, the one or more user devices are configured to collect one or more parameters of the user, and (b) a server comprising a database, a transceiver and a processing unit, having one or more modules for processing the one or more parameters for determining a one or more suggestions. Further, the determined one or more suggestions are transmitted to the output unit of the one or more user devices via communication channel that connects the one or more user devices and the server.

### CROSS-REFERENCE TO RELATED APPLICATION

This application claims the benefit of Indian Provisional Application Ser. No. 202211043705, filed on Jul. 30, 2022, entitled “MENSTRUAL CYCLE BASED MULTI-DIMENSIONAL METHODOLOGY FOR PERSONALISED PREDICTIVE, PREVENTIVE AND CURATIVE CARE,” commonly assigned with this application and incorporated herein by reference in its entirety.

### FIELD OF THE DISCLOSURE

The present disclosure generally relates to a personalized skincare, haircare, personal care system with overall user's health and wellness guiding devices in a non-face-to face environment. More particularly, the present disclosure relates to a system and method for providing preventive, palliative and curative care using plurality of parameters.

### BACKGROUND

Menstrual cycle is a natural process during the lifetime of a woman. When menstruation hits women, their physical and chemical bodily parameters, particularly the hormone levels, change in their approximate 28 days cycle period. Experts say that these menstrual parameters are also associated with skin conditions viz breakouts, bloating, hirsutism, hair fall and other health parameters. The major menstrual parameters that affect the overall skin conditions are the hormones like estrogens, progesterone, testosterones, androgens, prostaglandins, and others.

In general, the menstruation comprises of four different phases, and the menstrual hormonal levels may differ from phase to phase and even between the phases. The hormonal fluctuations also vary from individual to individual and between various ages. The four phases are menstrual phase, follicular phase, ovulation phase and luteal phase. The menstrual phase is the shedding of blood where the level of estrogen and progesterone drops and the female body releases prostaglandins which is another type of hormone. The follicular phase is the pre-release of egg phase, where the level of estrogen starts increasing. This is the phase of time between the first day of the period and the ovulation and hence there is a slight overlap. After this phase under the increasing influence of estrogen the uterine linings build up back again and between the first and second week there is an impact on the skin scent. Hormone estradiol is secreted and so is Follicle Stimulating Hormone. The ovulation phase, otherwise known as fertilization phase or the egg release phase, where the estrogen reaches its peak and then drops shortly afterwards. At this time testosterone is also released. Next to the Ovulation is the Luteal phase, this is the phase that starts before the next phase of menstruation where the luteinizing hormone and follicle-stimulating hormone levels decrease. The ruptured follicle closes after releasing the egg and forms a corpus luteum, which produces progesterone. During most of this phase, the estrogen level is high, which start tapering off around week 3. Prostaglandin and Cortisol start getting produced.

On the other hand, most of these hormones are associated with the sebaceous glands. An important hormone is the androgen hormone which has the capacity to bind with the sebaceous gland to produce more sebum. This may induce the skin to be oily and makes the skin prone to acne. Further, the progesterone is also responsible for dull skin, where abnormal or altered levels of progesterone affect the skin health. In the similar way when estrogen is more it leads to glowing and youthful skin as it helps to build up collagen and elastin matrix that helps in overcoming wrinkling and signs of ageing on the skin.

The breakout of acne and other skin related problems can also be cyclic according to the menstrual cycle. People with skin problem often visit doctor to get free from the skin & health related issues such as acne rosacea, dull and dry skin, freckles, melasma, bloated face and eyes, dysmenorrhea, cystic ovaries, hirsutism etc., for consultation, where this kind of consultations are expensive and requires multiple visits. There are a few mobile based applications that have been developed to provide consultation based on questionnaires. For example, several prior arts disclose about a system or a method for recommending the skin care routine based on the external parameters and questionnaires.

However, none of the prior art provides an in-home and cost-effective solution for providing or suggesting personalized skin care and well-being on a daily basis, based on the changes related to the menstrual parameters, as the skin parameters change significantly during the period of the cycle as a result of these hormonal changes.

Hence, there is a need for novel, cost-effective and in-homebased solution that provides skin care and user's wellness/health related suggestions for preventive, palliative and curative care.

### SUMMARY

The primary objective of the present disclosure is to develop a novel system, and method for providing personalized predictive, preventive, palliative and curative care for a user.

Another objective of the present disclosure is to develop an in-home personalized or customized system and method for pre-empting or predicting the user's health and future graph of the illnesses and other impacts related to hormonal variations and recommending suggestions in advance.

Yet another objective of the present disclosure is to provide a cost-effective system, and method for providing suggestions by correlating the menstrual cycle parameters with various other parameters.

Still another objective of the present disclosure is to provide a novel system for providing personalized skin care routine based on the menstrual parameters.

Further objective of the present disclosure is to improve the quality of life of the user by suggesting appropriate predictive, preventive, palliative, and curative skin care in a timely manner.

Another objective is to use technology to provide a methodology for providing a daily care recommendation and regimen based on the day of the monthly cycle and other parameters towards accentuated results which is hitherto not being done by the dermatologists and other specialists.

Another objective is to provide a kit having base cosmetic formulation, where the user is allowed to mix recommended amount of one or more active ingredients, in the formulation already provided, based on the suggestions of the system of the present disclosure.

To achieve the above objectives, the present disclosure provides with a method for providing one or more suggestions, where the method being performed by a system comprising one or more user devices comprising one or more sensors, an input unit, an output unit and a transceiver, and a server comprising a database, a transceiver, and a processing unit having one or more modules, where the one or more user devices are communicatively coupled to the server via a communication network.

The method comprising the steps of receiving, by the server, a request from the user via the one or more devices to provide the one or more suggestions, extracting by the server a corresponding one or more parameters of the user through one or more user devices which is stored in the database of the server, processing, by the server, a first set of parameters to generate a first input, processing, by the server, a second set of parameters to generate a second input, processing, by the server, a third set of parameters, the first input, and the second input to generate one or more suggestions, and displaying by the server using the output unit of the one or more user devices the one or more suggestions relating to predictive, preventive, palliative and curative care.

According to another aspect of the present disclosure, a personalized system for providing one or more suggestions to a user, the system comprising, (a) one or more user devices comprising one or more sensors, an input unit, an output unit and a transceiver, the one or more user devices are configured to collect one or more parameters of the user, and (b) a server comprising a database, a transceiver and a processing unit. The processing unit comprises one or more modules that are configured to process the one or more parameters for determining one or more suggestions. Then the determined one or more suggestions are transmitted to the output unit of the one or more user devices, where the server and the one or more user devices are configured to be communicatively coupled via a communication network. The determined one or more suggestions are predictive, preventive, palliative and/or curative care.

According to the present disclosure, the one or more sensors are selected from the group consisting of: an image sensor, a body temperature sensor, a location sensor, Heart rate monitor, oximetry sensor, accelerometer, ECG sensor, UV sensors, electrodermal activity sensor, skin conductance sensor and a combination thereof.

According to one embodiment of the present disclosure, the first set of parameters, the second set of parameters, and the third set of parameters are selected from the group comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy and childbirth, and combinations thereof. In one embodiment, the first set of parameters, the second set of parameters and the third set of parameters is selected from the one or more parameters obtained from the user via one or more user devices.

According to one embodiment of the present disclosure, one or more parameters collected through the one or more user devices, comprising age, skin type, menstrual cycle information, body temperature change, ethnicity, location, goals, sensitivity, medical information/conditions, lifestyle, occupation, skin condition, physical activity, stress, sleep, preferences, face mapping, pregnancy and childbirth, and combinations thereof. More specifically, the one or more parameters obtained from the user through various means corresponds to obtaining one or more of but not limited to, (a) parameter corresponds to a menstrual cycle, hormones etc., (b) a parameter corresponds to an user's skin type and features associated with it, (c) a parameter corresponds to location details of the user to predict the upcoming weather conditions for a predetermined period, (d) a parameter corresponds to lifestyle information, and other information like (e) a parameter corresponds to an ethnicity of the user, (f) a parameter corresponds to an age of the user, (g) a parameter corresponds to goals of the user (h) a parameter corresponds to medical issues of the user particularly relating to the beauty and wellness besides basic information of the user, (i) a parameter corresponds to sensitivity, (j) a parameter corresponds to occupation, (k) a parameter corresponds to preferences, (l) a parameter corresponds to pregnancy and childbirth, and combinations thereof.

According to one embodiment of the present disclosure, the one or more sensors in the one or more user devices collects menstrual cycle monitoring parameter. In another embodiment, the one or more user devices comprising the input unit that is configured to collect the menstrual parameters through a list of questionnaires displayed in the one or more user devices.

According to one embodiment of the present disclosure, the one or more user devices comprising the input unit collects at least one skin type information of the user through a list of questionnaires displayed in the one or more user devices. According to another embodiment, the one or more sensors in the one or more devices is configured to provide user's skin type parameters.

In another embodiment, the one or more user devices comprising the input unit where the input unit enables the user to enter or upload the result of a standard paper test done for the skin type parameter. In one embodiment, the skin type parameter corresponds to oily, dry, normal, combinations and finer sub-categories and combinations thereof.

According to one embodiment of the present disclosure, the one or more user devices comprising the input unit collects at least one lifestyle parameter or at least one basic parameter of the user through a list of questionnaires displayed in the one or more user devices.

According to one embodiment, the one or more sensors in the one or more user devices is configured to collect the location details of the user and is also configured to predict the upcoming weather for a predefined time period.

According to one embodiment of the present disclosure, the output unit of the one or more user device delivers the determined one or more suggestions corresponding to a predictive, preventive, palliative, and curative suggestions through a visual display, an audio-based suggestion, a suggestive pop-up message on user's mobile, personal computer, e-device screen from time to time or combinations thereof.

According to one embodiment of the present disclosure, the user is a female. According to another embodiment of the present disclosure, the menstrual parameters are excluded when the user is a male.

According to one embodiment of the present disclosure, the one or more user devices comprises one or more user interfaces corresponding to a male user and a female user.

Thus, the advantages of the present disclosure relating to but not limited to are, a novel, cost-effective, system and method that accurately predicts and provides the predictive, preventive, palliative, and therapeutic suggestions to the user daily/in real-time.

### DESCRIPTION OF THE DRAWINGS

The foregoing and other features and advantages of the disclosure will be more fully understood from the following description made with reference to the drawings.

FIG. 1a illustrates an overall block diagram representing a system for providing predictive, preventive, palliative, and curative care suggestion according to one exemplary embodiment of the present disclosure;

FIG. 1b illustrates a processing device and its components for determining the suggestion to a user according to one exemplary embodiment;

FIG. 2a-2c illustrate a diagrammatic representation of collection of various information through various means according to one exemplary embodiment the present disclosure;

FIG. 3a-b illustrates a flow chart representing the method for providing predictive, preventive, palliative, and curative care, according to one exemplary embodiment of the present disclosure.

FIG. 4 illustrate a flow chart representing an exemplary method to identify the skin type according to the disclosure;

FIG. FA illustrates a face mapping with indicative organ involvement according to the embodiments of the disclosure;

FIG. FB illustrates wrinkle face-mapping according to the embodiments of the disclosure;

FIG. FC illustrates pigmentation mapping of the face according to the embodiments of the disclosure;

FIG. FD illustrates acne mapping of the face according to the embodiments of the disclosure; and

FIG. FE illustrates zone wise division of the face according to the embodiment of the disclosure;

### DETAILED DESCRIPTION

Aspects of the present disclosure are best understood by reference to the description set forth herein. All the aspects described herein will be better appreciated and understood when considered in conjunction with the following descriptions. It should be understood, however, that the following descriptions, while indicating preferred aspects and numerous specific details thereof, are given by way of illustration only and should not be treated as limitations. Changes and modifications may be made within the scope herein without departing from the spirit and scope thereof, and the present disclosure herein includes all such modifications.

Several aspects of the present disclosure are disclosed herein. It is to be understood that these aspects may or may not overlap with one another. Thus, part of one aspect may fall within the scope of another aspect, and vice versa. Each aspect is illustrated by a number of embodiments, each of which in turn, can include one or more specific embodiments. It is to be understood that the embodiments may or may not overlap with each other. Thus, part of one embodiment, or specific embodiments thereof, may or may not fall within the ambit of another, or specific embodiments thereof, and vice versa.

A broad framework of the principles will be presented by describing various embodiments of this disclosure using exemplary aspects. The terms “one embodiment” or “an embodiment” means that a particular feature, structure, material, or characteristic described in connection with the embodiment is included in at least one embodiment of the disclosure. For clarity and ease of description, each aspect includes only a few embodiments. Different embodiments from different aspects may be combined or practiced separately, to design a customized process or product depending upon application requirements. Many different combinations and sub-combinations of a few representative processes or structures shown within the broad framework of this disclosure, that may be apparent to those skilled in the art but not explicitly shown or described, should not be construed as precluded.

The present disclosure relates to a novel system, and method for providing predictive, preventive, palliative and curative care for the user.

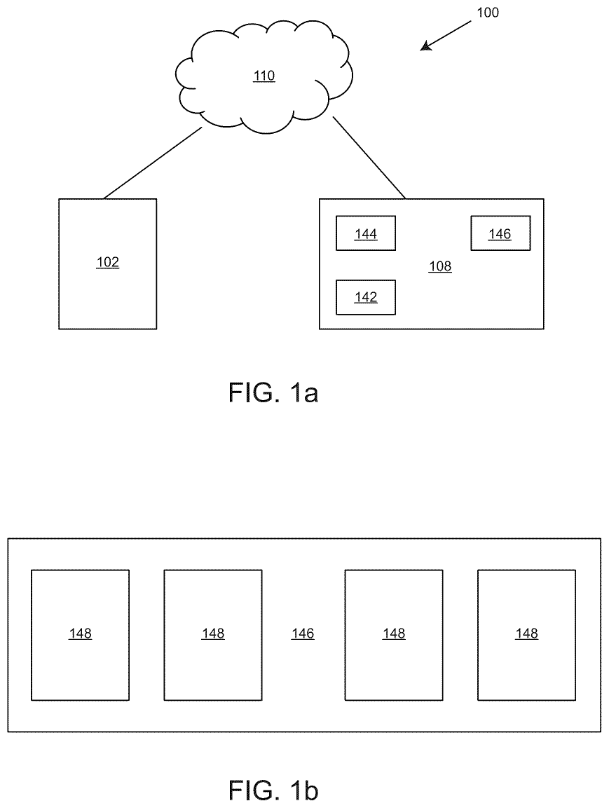

According to one aspect of the present disclosure, FIG. 1a illustrates a personalized system (100) for providing one or more suggestions to a user. The system (100) comprising, (a) one or more user devices (102) comprising one or more sensors, an input unit, an output unit, and a transceiver. The one or more user devices (102) are configured to collect one or more parameters of the user, and the system (100) further comprises a server (108) comprising a database (144), a transceiver (142) and a processing unit (146).

The processing unit (146) comprises one or more modules (148) as illustrated in FIG. 1b, where the one or more modules (148) are configured to process the one or more parameters for determining one or more suggestions. Then the determined one or more suggestions are transmitted to the output unit of the one or more user devices (102), where the server (108) and the one or more user devices (102) are configured to be communicatively coupled via a communication network. The one or more suggestions are predictive, preventive, palliative and curative care.

According to the present disclosure, the one or more sensors are selected from the group consisting of: an image sensor, a body temperature sensor, a location sensor, Heart rate monitor, oximetry sensor, accelerometer, ECG sensor, UV sensors, electrodermal activity sensor, skin conductance sensor and a combination thereof.

According to one embodiment of the present disclosure, the first set of parameters, the second set of parameters, and the third set of parameters are selected from the group comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy and childbirth, and combinations thereof. In one embodiment, the first set of parameters, the second set of parameters and the third set of parameters is selected from the one or more parameters obtained from the user via one or more user devices.

According to one embodiment of the present disclosure, the one or more parameters collected through the one or more user devices comprise age, skin type, menstrual cycle information, body temperature change, ethnicity, location, goals, sensitivity, medical information/conditions, lifestyle, occupation, skin condition, physical activity, stress, sleep, preferences, face mapping, pregnancy and childbirth, and combinations thereof.

More specifically, the one or more parameters obtained from the user through various means corresponds to obtaining one or more of but not limited to, (a) parameter corresponds to a menstrual cycle, hormones etc., (b) a parameter corresponds to an user's skin type and features associated with it, (c) a parameter corresponds to location details of the user to predict the upcoming weather conditions for a predetermined period, (d) a parameter corresponds to lifestyle information, and other information like (e) a parameter corresponds to an ethnicity of the user, (f) a parameter corresponds to an age of the user, (g) a parameter corresponds to goals of the user (h) a parameter corresponds to medical issues of the user particularly relating to the beauty and wellness besides basic information of the user, (i) a parameter corresponds to sensitivity, (j) a parameter corresponds to occupation, (k) a parameter corresponds to preferences, (l) a parameter corresponds to pregnancy and childbirth, and combinations thereof.

According to one embodiment of the present disclosure, the one or more user devices (102) comprising one or more sensors that is configured to collect menstrual parameters. In one example, the menstrual parameters like menstrual parameters are collected through information Artificial Intelligence built smart-watch/wearable device or through an android/ios installed application, through body temperature monitoring system and devices and other means that are known in the prior art. According to another embodiment, the menstrual parameter is collected through a series of user-friendly questionnaires displayed in the one or more user devices (102) through the input unit. In yet another embodiment, the menstrual parameter is collected through a series of user-friendly questionnaires in a paper where the user has to feed the answers manually to the smart device. The menstrual cycle comprising but not limited to are hormones like estrogen, progesterone, testosterone, prostaglandins, and sebaceous, hormonal level in a monthly cycle of a female through blood tests, day of the cycle, and other related parameters. In case of questionnaire, the user is guided to a next level interface.

According to an embodiment of the present disclosure, the one or more user devices (102) comprising one or more sensors which is configured to collect location parameter of the user. For example, the location parameter obtains input from in built GPS Tracker in the smart device or an android or ios based app that is installed in the user's smart device. This location parameter is used to access and predict the upcoming weather parameter for a period of time, (e.g., 7 days). The weather parameter also includes the information about the weather details like air quality index (AQI), humidity, and other information like dust particles, microbes in the air etc. The one or more user devices (102) or another smart device is used to get an indication of the stress levels of the user.

According to an embodiment of the present disclosure, one or more user devices (102) comprising the input unit is configured to collect the lifestyle parameter through a set of questionnaires displayed in the one or more user devices (102). The lifestyle and basic parameters relate, but not limited to, the age of the user, smoking, computer usage, medications, ethnicity, sun exposure, environment, stress level, diet, sleep pattern, lifestyle, alcohol consumption, occupation, psychological state, and other parameters.

According to an embodiment of the present disclosure, the one or more user devices (102) comprising one or more sensors that is configured to collect information regarding user's facial skin parameters. In one embodiment, the sensor is an artificial intelligence-based image sensor. According to the present disclosure, the artificial intelligence-based image sensor is a camera, where the camera captures the user's face and processes the obtained image to identify the user skin types, acne, blemishes, and other skin issues and parameters. The general skin types of the user comprising dry skin, normal skin, oily skin, combination skin or finer sub-categories and/or combinations thereof. In one embodiment, the other facial skin parameters, or information like, but not limited to, acne breakouts, marks, dark spots, and fine lines can also be identified. According to another embodiment of the present disclosure, the one or more user devices (102) comprising the input unit is configured to collect and obtain user's facial skin parameters are obtained through a questionnaire, where the user is allowed to answer via input unit. In another embodiment, the one or more user devices (102) comprising the input unit where the input unit enables the user to enter or upload the result of a standard paper test done for the skin type parameter.

In one embodiment, the skin type parameter corresponds to oily, dry, normal, combinations and finer sub-categories and combinations thereof. According to the present disclosure, the input unit of the one or more user devices (102) subjectively collect at least one skin type parameter of the user where the user's skin type is oily, normal, dry or combination, or finer sub-categories and/or combinations thereof. The input unit can take input derived through a standard tissue paper test that can be compared with the sensor results for cross confirmation or the device collects fine sub-categories of the skin types such as oily+, oily++ or oily+++ or Normal to dry etc., through a questionnaire which can further be compared with the AI image sensor results for cross confirmation.

According to the present disclosure, the one or more sensors, input unit of the one or more user devices (102) can be utilized to generate the skin type data, in which the one or more user devices (102) are connected to a pc through a communication application. This application having an inbuilt software that can be related through image sensors to analyze and calculate the skin oil, moisture levels, skin type, skin tone and condition viz acne, pigmentation, wrinkles etc., but not limited to the above mentioned.

The system further collects data related to facial skin using questionnaires, smart watch, or an application-controlled device. The facial skin image analyzer includes several units to analyze the skin type information like moisture calculation (e.g., high/normal/low), oily (e.g., high/normal/low), skin tone (e.g., light/normal/dark), acne (e.g., severe/moderate/less) and post analyses correlate this skin type information with the location on the correlation matrix. Further a predictive, preventive, palliative, and/or curative suggestion message is periodically generated based on the altering skin factors/environment influence (which includes humidity, temperature variations etc.,).

According to the present disclosure, an optional structured one or more user devices (102) to obtain skin type parameter to subjectively collect the information of the skin type of the user whether oily, normal, dry or combination or finer sub-categories and/or combinations thereof, through the standard tissue paper test, or a AI senor image analyzing process where the result of the tissue paper test is compared with the artificial intelligence-based image sensor results for cross confirmation purpose.

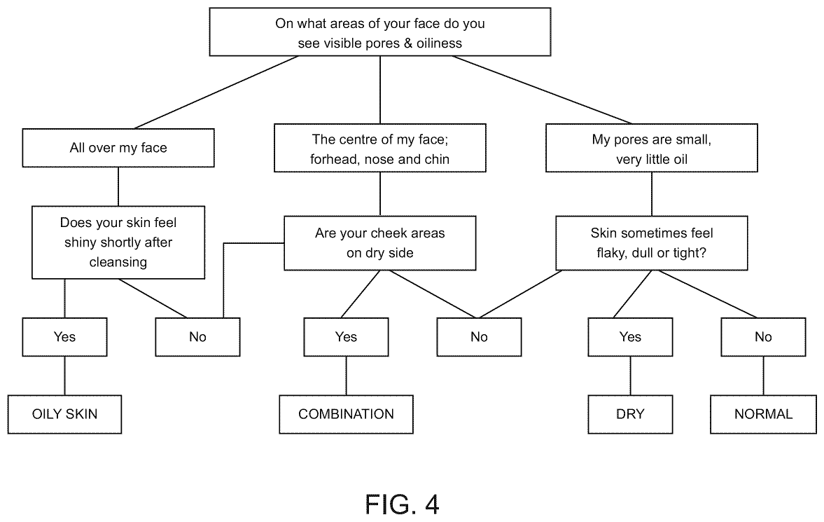

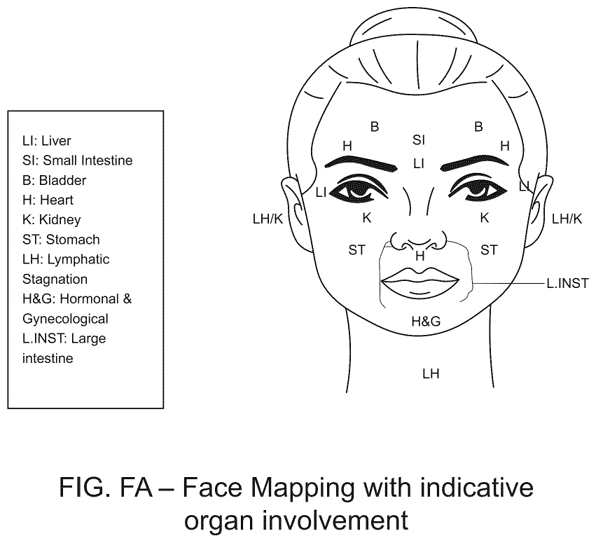

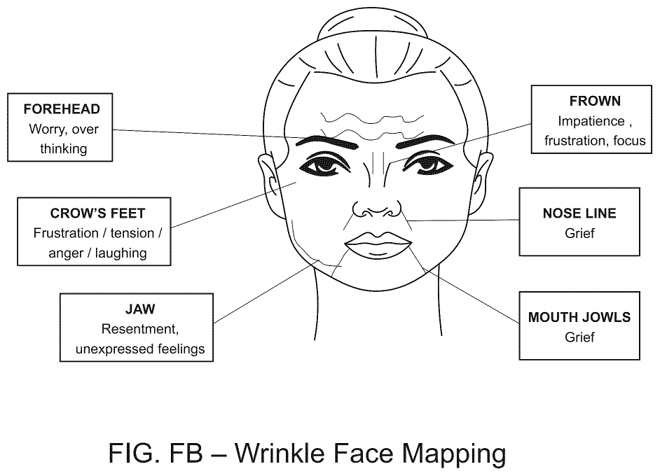

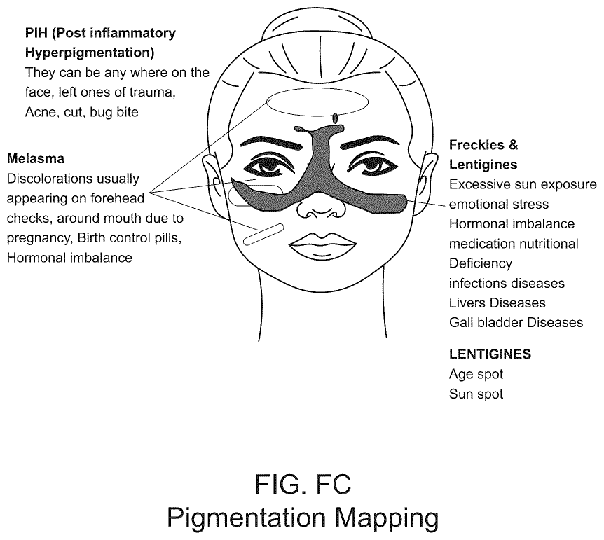

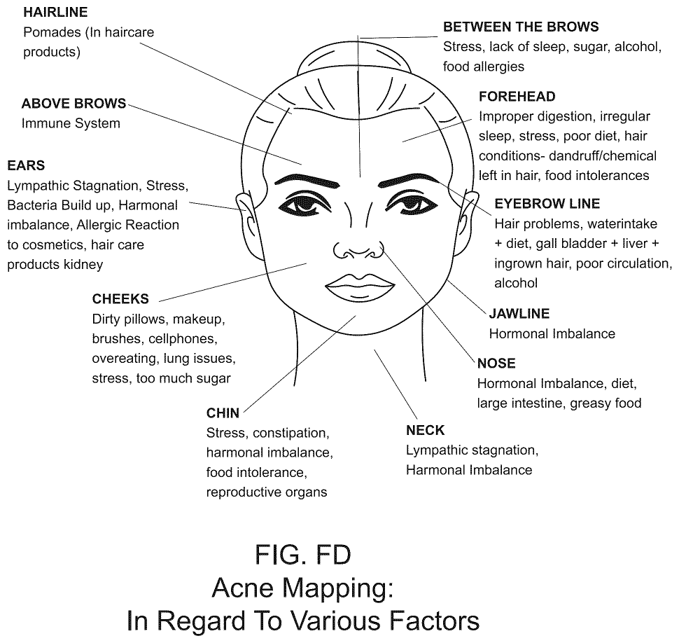

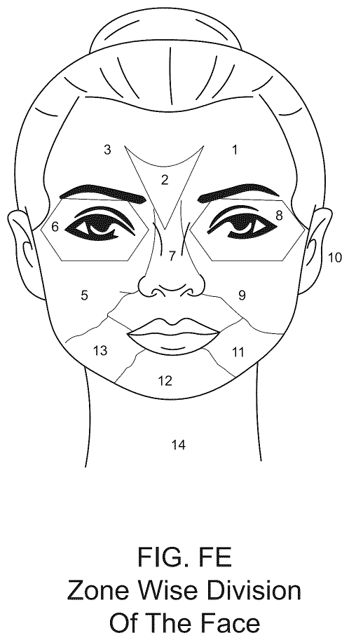

FIG. 4 illustrates the flowchart representing an exemplary method of processing the skin parameter to analyze the skin type information of the user according to the questionnaire. Based on the response provided by the user the system can determine the type of skin selected from normal, dry, combination and oily, or finer sub-categories and/or combinations thereof.

The processing unit (146) comprising one or more modules (148) analyses the visible pores and oil areas in the user's face based on three features. In the first feature, when the pores and oil are visible all over the face, then the module analyses and provides output as oily skin based on the criteria shinier skin after cleansing is positive. In the second feature, when the pores are small and very less oil is observed on the user's face, then the one or more modules (148) determines the skin type as dry based on skin type criteria like flaky, dull and tightness of the skin is positive. In other case, when the pores are small and very less oil is observed on the user's face, then the one or more modules (148) determines the skin type as normal based on the skin type criteria like flaky, dull and tightness of the skin is negative. In the third feature, when the pores and oil are observed only in the center of the face, forehead, nose, and chin, then the one or more modules (148) determines the skin type as combination skin, based on the skin type criteria like dry cheek area is positive.

According to the present disclosure, the skin type information is also generated by the face mapping technique (includes acne mapping, wrinkle mapping, pigmentation mapping, and not limited to the one mentioned but also covers the other mapping techniques available in the prior art), relating the various skin conditions with the internal problems so that well-being regime can be planned and guided to the user in a timely manner. Pop up messages are also curated in the individual case related to the doctor consultation is required, and not limited to other suggestive measures to be guided if needed as far as its possible without the subjective presence of the user and can be done online/without face-to face environment.

### Face Mapping:

Face mapping is being followed since ancient times in China. The latest version of face mapping is made with the combination of Chinese face mapping and scientific knowledge and is widely used in predictive way to detect face issues along with internal issues so that the person can be directed for the right treatment path.

In the present disclosure, scientific face mapping tool is used which is created by combining ancient and modern knowledge available thereby constructing a hybrid tool that can work as a predictive, preventive, palliative, and curative care tool for the user who is taking a suggestive feedback on using it.

Referring to FIGs. FA, FB, FC, FD for understanding the areas involved in face mapping. The figures have elaborative view for face mapping, general based upon Chinese mapping (FIG FA), Wrinkle Face mapping (FIG. FB), pigmentation face mapping (FIG. FC), and Acne Face Mapping (FIG. FD).

The face is once again split into zones (referring to FIG. FE) and analyzed. Combining this analysis with a lifestyle questionnaire allows determination of reasons for various skin issues, ranging from acne to rosacea and eczema as an example.

The zones defined in FIG. FE is explained in detailed below.

Zone 1 and 3: The forehead

Any issues here indicate a potential problem with the bladder or digestive system. Breakouts can mean a poor diet and lack of water. Congestion can also signal improper makeup or shampoo removal, or products that are clogging your pores. There's even a name for this condition: acne cosmetica.

Zone 2: Between the eyebrows

The middle of your forehead is linked to your liver. Pimples or oiliness here suggest a diet overhaul.

Avoiding excess alcohol, rich foods, and late-night eating is advisable along with checking for food allergies, particularly a lactose intolerance.

Zone 4 and 10: The ears

Kidney problems can show up here. People with hot ears are advised to up their water intake and reduce caffeine, alcohol, or salt consumption. Other irritations may be related to cellphone use or jewelry allergies.

Zone 5 and 9: The cheeks

Linked to the respiratory system, those who smoke or have allergies may notice the likes of broken capillaries, hyperpigmentation, or general congestion. These may also be a result of comedogenic cosmetic ingredients, gum or tooth problems, or bacteria from cellphones.

Bacteria is one of the biggest contributors to acne, and studies show you should be particularly aware of your phone's hygiene. These devices harbor a high number of bacteria Trusted Source, some of which could cause diseases.

Zone 6 and 8: The eyes

Kidneys are also connected to the eyes. Dark circles and puffiness can be a sign of dehydration or an imbalanced diet. But allergies, poor lymph flow, and kidney stress can also be contributing factors.

Zone 7: The nose

Broken capillaries around the nose can have a simple cause, ranging from heavily squeezing pimples to the environment or genetics. But if the nose itself is redder than usual, this may be a sign of high blood pressure as it's linked to the heart.

Zone 12: The chin center

A breakout here can relate to a hormonal imbalance, too. The middle of your chin also corresponds with the small intestine, so dietary problems or food allergies may be the cause of any issues.

Zone 12A: The upper lip

The space above your lip is associated with the reproductive system. Pigmentation or excessive hair growth here is thought to be a result of a hormonal imbalance.

Changing hormone levels can affect melanin production, leading to hyperpigmentation. And increased production of hormones called androgens can result in unwanted hair growth—also known as hirsutism.

Zone 11 and 13: The jawline

Dental work can kick off a breakout along the sides of your chin. But so can hormones as this area is related to the ovaries. Pimples here can also be influenced by a failure to properly remove makeup, irritating or pore-clogging ingredients in cosmetics, and constant touching of the area.

The one or more user devices (102) further comprising a transceiver for sending the one or more parameters to the server (108), where the one or more parameters are obtained through the one or more sensors and the input unit of the one or more user devises (102).

The one or more user devices (102) is communicatively coupled to the server (108) via a communication network (110), where the one or more parameters are transmitted through the transceiver to the server (108) via a wireless connection. Suitable examples of wireless communication comprise but not limited to are Bluetooth, near field communication, infrared (IR), cellular networks, Zigbee, z-wave, LoRa, satellite communication, Li-fi, WiMAX, wi-fi, radio waves and others wireless communications that are well known in the art.

The one or more parameters that are transmitted to the server (108) is received by the transceiver (142) and the one or more parameters are stored in the database (144) of the server (108).

The processing unit (146) of the server (108) comprising one or more modules (148) where the one or more parameters from the database (144) is extracted and further processed to one or more suggestions corresponding to predictive, preventive, palliative and curative care. In one embodiment, the system and method of the present disclosure comprises only one processing unit (146) with only one module for processing the one or more parameters. In another embodiment, the system and method of the present disclosure comprises only one processing unit (146) with one or more modules for processing the one or more parameters. In another embodiment, the system and method of the present disclosure comprises more than one processing units (146) with one or more modules respectively for processing the one or more parameters.

In one embodiment, the one or more modules (148) extracts and processes the first set of parameters, the second set of parameters and the third set of parameters separately to generate one or more suggestions. In another embodiment, the one or more modules (148) extracts and processes the first set of parameters, the second set of parameters and the third set of parameters simultaneously to generate one or more suggestions.

In another embodiment, the one or more modules (148) of the processing unit (146) of the server (108) comprises a first module to extract and process the first set of parameters, a second module to extract and process the second set of parameters and a third module to extract and process the third set of parameters. In another embodiment, the one or more modules (148) comprises more than three modules to extract and processes one or more parameters according to the number of sets of the parameters. In another embodiment, the server (108) comprises more than one processing unit (146) to process the one or more parameters to generate the one or more suggestions.

The one or more modules (148) of the processing unit (146) of the server (108) processes the obtained information through a multi-dimensional correlation matrix.

The creation of the multi-dimensional correlation matrix involve gathering parameters from the user through one or more user devices (102) such as AI based image sensor for picture and video capturing, periodic blood tests information through the app with the tied-up laboratories/individual selection of the user, through smart-watch/wearable/non-wearable device tracking, through series of structured, precise and user friendly questionnaire(s) for gathering various demographic, psychographic and other information on user lifestyle, stress, job profile, goals to achieve etc., from time to time. Further, the system comprises one or more additional processing unit to process the lifestyle related parameters (not shown) uses the multi-dimensional correlation matrix to provide one or more suggestions based on the parameters.

The multidimensional correlation matrix compares the one or more various information selected from but not limited to age, weather, skin type (e.g., oily, normal, dry, combination, or finer sub-categories and/or combinations thereof) skin sensitivity (e.g., sensitive and non-sensitive), UV index, location/living environment, ethnicity, weather information like air pollution, temperature, humidity, dust/fine dust conditions, and lifestyle details including alcohol, nutritional/eating habits, stress, mood, psychological state (e.g., calm, normal, energetic, not so sad, stressed), amount of sleep(e.g., more than 8 hrs, 8 hrs or less than 8 hrs), user preferences (such as perfumed/non-perfumed, herbal etc.), sedentary lifestyle, exercise, blue light, UV index, outdoor exposure time/activity time, occupation of the user and the user preferences like perfumes, cosmetics and user goals. In one embodiment, the multidimensional correlation matrix is already trained with one or more suggestions for the information. Based on the training data, the system automatically determines and provide one or more suggestions to the user through feedback. The one or more suggestions determined by the one or more modules (148) of the processing unit (146) is transmitted from the server (108) through the transceiver (142) to the one or more user devices (102), where the transceiver of the one or more user devices (102) receives the one or more suggestions and delivers the output in the form of one or more suggestions through a visual display, an audio-based suggestions, and a suggestive pop-up message on the one or more user devices (102).

The processing unit provides one or more suggestions relating to the type of cosmetic & wellbeing regime needed depending upon the menstrual cycle, predictive moisture level (high, low, normal), skin condition index-wrinkle index, trouble index by analyzing the photograph of the skin of the user to create a customized hybrid based collaborative skincare, personal care, wellbeing planner that can provide a facial health care system on the big data and also simultaneously help in detection/pre-emption of prediabetes, insulin resistance, vaginal health issues, infertility so that the user can be directed for the doctor consultation well in time.

According to one embodiment, the one or more suggestions are predictive, preventive, palliative and curative care. The one or more suggestions are delivered through a visual display, an audio-based suggestion, and/or a prompt like suggestive pop-up message on user's mobile, personal computer, e-device screen from time to time.

According to the present disclosure, the one or more suggestions correspond restorative skin care, preventive skin care, cosmetic skincare, haircare, lipcare, mood/emotional upliftment, menstrual parameters like menstrual mood swings, menstrual cramps, pain management, vaginal health, prediabetic state, infertility related issues and other aspects of well-being and combinations thereof.

According to the present disclosure, the system and method of the present disclosure predicts the warning signs of menstrual mood swings, menstrual cramps, and its effect on skin. The system also provides preliminary prediction regarding the potential major complications associated with onset of diabetes, prediabetes, insulin resistance, polycystic ovary syndrome (PCOS) that leads to infertility, hormonal imbalances, stress, and other factors that could lead to infertility.

According to the present disclosure, the one or more user devices (102) are but not limited to are smart phone, smart watch, computer, laptop, and other digital devices.

According to the present disclosure, the user is a female. According to the present disclosure the system excludes the menstrual parameters, pregnancy and childbirth parameters when the user is a male.

According to one embodiment of the present disclosure, the one or more user devices (102) comprises one or more user interfaces corresponding to a male user and a female user.

According to another aspect of the present disclosure, a method for providing one or more suggestions, where the method being performed by a system (100) comprising one or more user devices (102) comprising one or more sensors, an input unit, an output unit and a transceiver, and a server (108) comprising a database (144), a transceiver (142), and a processing unit (146) having one or more modules, where the one or more user devices (102) are communicatively coupled to the server (108) via a communication network.

The method comprising the steps of receiving, by the server (108), a request from the user via the one or more user devices (102) to provide the one or more suggestions, extracting by the server (108) a corresponding one or more parameters of the user through one or more user devices (102) which is stored in the database (144) of the server (108), processing by the one or more modules (148) in the server the one or more parameters comparing and correlating the by the server (108) and for determining one or more suggestions related to the predictive, preventive, palliative and curative care, and displaying by the server using the output unit of the one or more user devices (102) the one or more suggestions relating to predictive, preventive, palliative and curative care.

According to the present disclosure, the one or more sensors are selected from the group consisting of: an image sensor, a body temperature sensor, a location sensor, Heart rate monitor, oximetry sensor, accelerometer, ECG sensor, UV sensors, electrodermal activity sensor, skin conductance sensor and a combination thereof.

According to one embodiment of the present disclosure, the first set of parameters, the second set of parameters, and the third set of parameters are selected from the group comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy and childbirth, and combinations thereof.

According to one embodiment of the present disclosure, the one or more parameters collected through the one or more user devices (102) comprising age, skin type, menstrual cycle information, body temperature change, ethnicity, location, goals, sensitivity, medical information/conditions, lifestyle, occupation, skin condition, physical activity, stress, sleep, preferences, face mapping, pregnancy and childbirth, and combinations thereof. More specifically, the one or more parameters obtained from the user through various means corresponds to obtaining one or more of but not limited to, (a) parameter corresponds to a menstrual cycle, hormones etc., (b) a parameter corresponds to an user's skin type and features associated with it, (c) a parameter corresponds to location details of the user to predict the upcoming weather conditions for a predetermined period, (d) a parameter corresponds to lifestyle information, and other information like (e) a parameter corresponds to an ethnicity of the user, (f) a parameter corresponds to an age of the user, (g) a parameter corresponds to goals of the user (h) a parameter corresponds to medical issues of the user particularly relating to the beauty and wellness besides basic information of the user, (i) a parameter corresponds to sensitivity, (j) a parameter corresponds to occupation, (k) a parameter corresponds to preferences, (l) a parameter corresponds to pregnancy and childbirth, and combinations thereof.

According to one embodiment of the present disclosure, the sensor in the one or more user devices (102) collect menstrual cycle parameter. In another embodiment, the one or more user devices (102) comprising the input unit that is configured to collect the menstrual parameters through a list of questionnaires displayed in the one or more user devices (102). In another embodiment, the sensor as well as the input unit of the one or more user devices are collecting the menstrual parameters at the same time. The menstrual cycle parameter is obtained from questionnaires displayed on the inbuilt android/ios applications and/or smart watch tracker/wearable devices. The hormonal information is obtained for an optional advanced hormonal device in built in the app where user gets an option to either schedule the hormonal assay at a defined periodicity or log the test reports by themselves after getting it done preferably from a recommended validated laboratory.

According to one embodiment of the present disclosure, the one or more user devices (102) comprising the input unit collects at least one skin type information of the user through a list of questionnaires displayed in the one or more user devices (102). According to another embodiment, the one or more sensors in the one or more devices is configured to provide user's skin type parameters. The user's skin type parameter is obtained using artificial intelligence-based image sensor. The preferable image sensor is camera which captures and analyses the skin to produce the parameter relating to the type of user's skin, acne, spots, wrinkles, blemishes etc. According to another embodiment of the present disclosure, the user's skin type is obtained using a simple tissue paper test, where the result obtained from the tissue paper test is correlated with the artificial intelligence-based image sensor. According to yet another embodiment of the present disclosure, the user's skin type information is obtained from the user through a questionnaire or a combination of tissue paper test, questionnaire, and artificial intelligence-based image sensor. In one embodiment, the questionnaire is displayed in the smart device like smart phone, where the user can choose or type their answers directly to the smart device. In another embodiment, the questionnaire is provided in paper form. In one embodiment, the skin type parameter corresponds to oily, dry, normal, combinations and finer sub-categories and combinations thereof.

According to one embodiment, the one or more sensors in the one or more user devices (102) is configured to collect the location details of the user and is also configured to predict the upcoming weather for a predefined time. The location detail of the user is obtained using GPS Tracker in-built in android/ios app/smart watch tracker/wearable devices and is used to determine and/or predict the weather parameters like moisture content in the air, and other information like dust particles, microbes in the air for the upcoming period.

According to one embodiment of the present disclosure, the one or more user devices (102) comprising the input unit collects at least one lifestyle parameter or at least one basic parameter of the user through a list of questionnaires displayed in the one or more user devices (102). According to the present disclosure, the lifestyle information, demographics, psychographic and other basic information is obtained using one or more devices, which displays a set of questionnaires that is to be filled by the user using the input unit. The lifestyle and basic information relate to the age of the user, smoking, computer usage, medications, ethnicity, ultraviolet (UV) or sun exposure, environment, stress level, diet, sleep pattern, alcohol consumption, occupation, psychological state, and other information. In one embodiment, the questionnaire is displayed in the smart device like smart phone, where the user can choose or type their answers directly to the smart device. In another embodiment, the questionnaire is provided in paper form.

According to one embodiment of the present disclosure, the output unit of the one or more user device delivers the one or more suggestions corresponding to a predictive, preventive, palliative, and curative suggestions through a visual display, an audio-based suggestion, a suggestive pop-up message on user's mobile, personal computer, e-device screen from time to time or combinations thereof.

Suitable one or more user devices (102) according to the present disclosure are but not limited to are smart phone, smart watch, computer, laptop, and other digital devices.

According to the present disclosure, the one or more suggestions are predictive, preventive, palliative and curative care. According to the present disclosure, the one or more suggestions are selected from the group comprising of restorative skin care, preventive skin care, cosmetic skincare, haircare, mood/emotional upliftment, menstrual parameters like menstrual mood swings, menstrual cramps, pain management, other aspects of healthcare, well-being and combinations thereof.

According to one embodiment of the present disclosure, the user is a female. According to another embodiment of the present disclosure, the menstrual parameters are excluded when the user is a male.

According to one embodiment of the present disclosure, the one or more user devices (102) comprises one or more user interfaces corresponding to a male user and a female user.

According to yet another aspect of the present disclosure, the customer or user is provided with a personalized or customized kit comprising a base cosmetic formulation such as a gel or cream either without the active ingredient(s) or with a minimal required quantity of the active ingredient(s). One or more of such active ingredients can be shipped later with instructions on their amounts required to be added to the base formulation as per the personalized need of the user. This minimizes the excess chemical usage and fine tuning it for the individual according to the individual need thereby additionally helping the environment. The customized wellbeing kit is additionally provided with few supplements like inositol, glutathione, primrose oil, aroma oils etc., These supplements can also be suggested as nutraceuticals not only help in improving the skin texture but also aid in improving few causes related to infertility. In other ways, the system and method of the present disclosure not only provides a customized way for cosmetics but broadly covers the 360° views for overall health and well-being of the user. Further, the system and method of the present disclosure helps in guiding and suggesting about the vaginal health, indicating the vaginal discharge timings and types to understand ovulation and helps in planning the pregnancy dates, and also help understanding women/female health issues like STD's, cervical erosion, cervical cancer etc., In another embodiment the suggested guided regime as per her individual device is represented as the user's customized skin care base formulations which can be added with the active ingredients when there are some additions required as per change in weather, location and lifestyle changes.

According to another embodiment of the present disclosure, the system and method provides a woman's wellbeing plan which also suggests a nearby dermatologist or gynecologist for some intervening procedures. Further, it is also provided with a guidance to direct for an expert or doctor's opinion within a facility to schedule a first consultation and then it leaves to the user whether to book an appointment with the expert health care professionals or to make a personal choice of physician.

The system and method according to the aspects/embodiments stated above will hereinafter be described specifically by exemplary embodiments. However, it should not be construed that the disclosure is limited to or by them. The exemplary embodiments compare at least one or more other parameters with the menstrual cycle parameter to provide preventive, palliative or curative care suggestions using three/multi-dimensional matrix correlation analysis.

According to one exemplary embodiment, the system and the method comprising the processing unit having one or more modules where the one or more parameters from the database are compared and correlated using multi-dimensional correlation matrix. The one or more modules using multi-dimensional correlation matrix compares the hormonal level on those particular days of the cycle with the corresponding skin condition to predict the upcoming indication e.g., acne breakout, skin dullness. Along with the indication the system will further provides preventive care regime to prevent the skin to have acne breakouts.

According to one exemplary embodiment, the one or more modules using multidimensional correlation matrix is used for comparing the hormonal level with the skin type and existing skin condition such as acne breakouts etc., to provide or suggest the appropriate care regimes including ointments or doctor's visit.

According to another exemplary embodiment of the present disclosure, the one or more modules using multidimensional correlation matrix for comparing hormonal changes with the skin condition with respect to each day in the menstrual cycle. Based on the correlation, one or more suggestions are determined, along with a preventive and curative treatment is suggested. The suggestions are, but not limited to, skin indications, menstrual health, mood and mood swing, emotional state, pain tolerance, urges and cravings, health conditions, and combination thereof.

According to another exemplary embodiment of the present disclosure, the one or more modules using multidimensional correlation matrix, compares and correlates various parameters and excludes menstrual parameters when the user is a male and provides one or more suggestions relating to their skin indications, haircare, wellness.—emotional state, health conditions and combination thereof.

According to another exemplary embodiment of the present disclosure, the one or more module using multidimensional correlation matrix is structured with the following steps. The first step for the formation of the multi-dimensional correlation matrix is to identify and categorize the user based on ethnicity viz Caucasian, Asian, Hispanic or African-American, African etc., and many other sub classifications done in old system classifications, but not limited to. Once the ethnicity segmentation is completed then other parameters or dimensions are superimposed sequentially to form the multi-dimensional matrix. The 1^{st} dimension of the matrix is the skin type viz Normal, Dry, Oily, Combination skin or finer sub-categories and/or combinations thereof is taken along with 2^{nd} dimension of the day of the menstrual cycle. Next dimension to be added to the matrix is Age to create a base matrix of the user. Separate base matrixes are created by taking the 2^{nd} dimension age viz<pre-menarche (A1), menarche-20 years(A2), 21-30 years(A3),31-40 years (A4), 40 years-pre-menopause(A5), Pre-menopause to menopause(A6) and >menopause (A7), In the next dimension of the matrix the weather information is taken depending upon the combination of the temperature and the relative humidity to create another dimension of the matrix in continuation of the ones made in the prior steps, further addition to the base matrix. Once the personalized matrix is identified in this multidimensional matrix system, then additional lifestyle factors including but not limited to sleep, alcohol, stress, sun exposure, excessive fatty food, excessive tea/coffee, unhealthy healthy diet, exercise, birth control pills, smoking, unhealthy weight, drug abuse and many more that comes under lifestyle which are not mentioned are added to fine tune the personalized profile under the matrix according to which the skin care, haircare, wellness and well-being guide is related to provide predictive, preventive, palliative, and curative regime to the individual user.

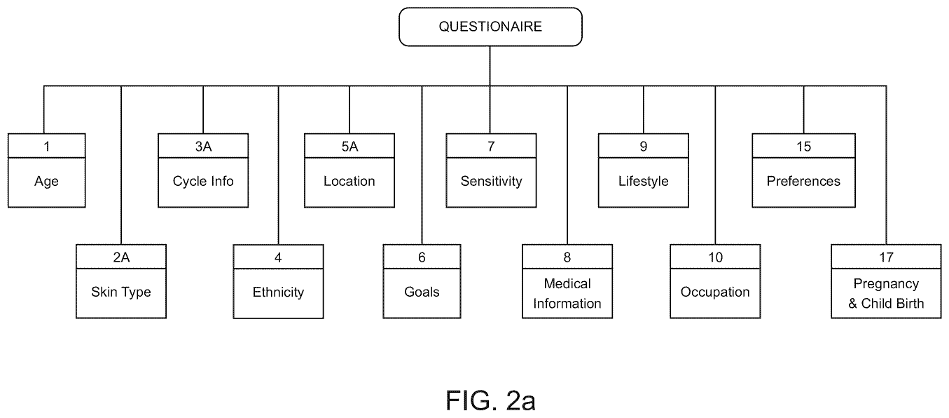

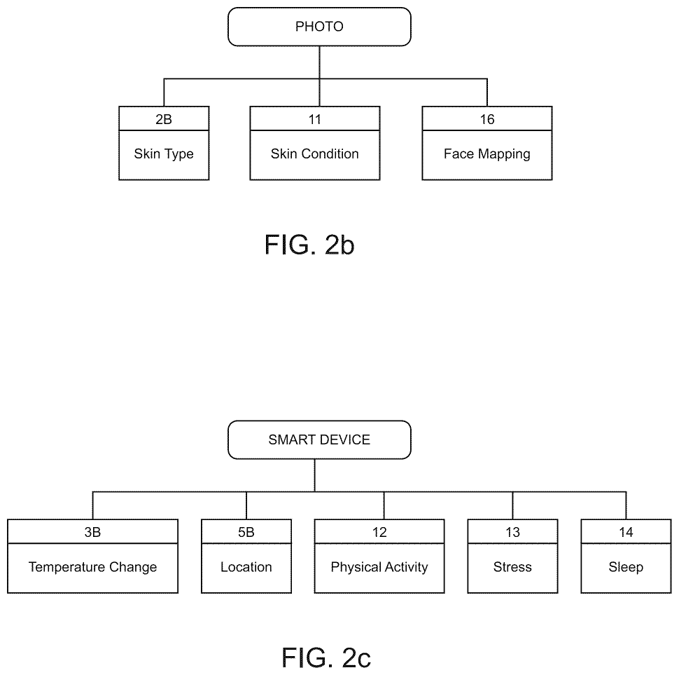

FIGS. 2a to 2c illustrate one or more devices and means for collecting the user's information according to one exemplary embodiment of the present disclosure. FIG. 2a illustrates a questionnaire that is used to collect information regarding age (1), skin type (2A), cycle information (3A), ethnicity (4), location (5A), goals (6), sensitivity (7), medical information (8), lifestyle (9), occupation (10), preferences (15) and pregnancy and childbirth (17). The skin type (2b), skin condition (11) and face mapping (16) are collected through photos as illustrated in FIG. 2b. FIG. 2c illustrates a smart device that is used to collect the information relating to body temperature change (3B), location (5B), physical activity (12), stress (13) and sleep (14).

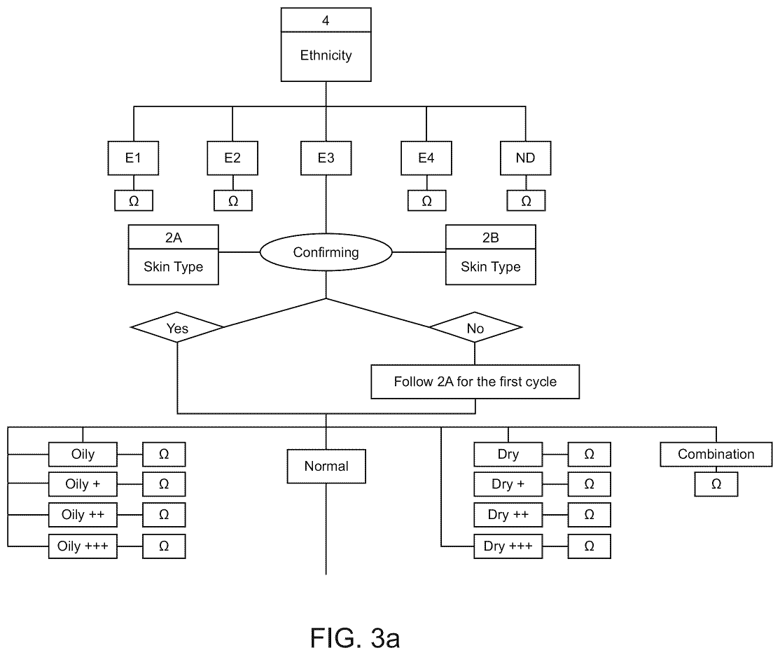

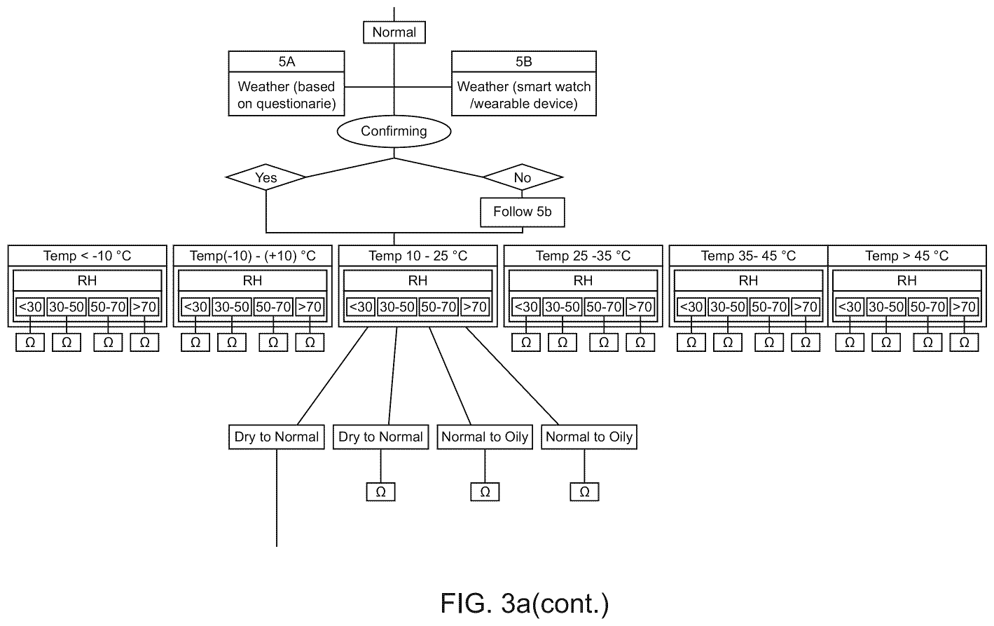

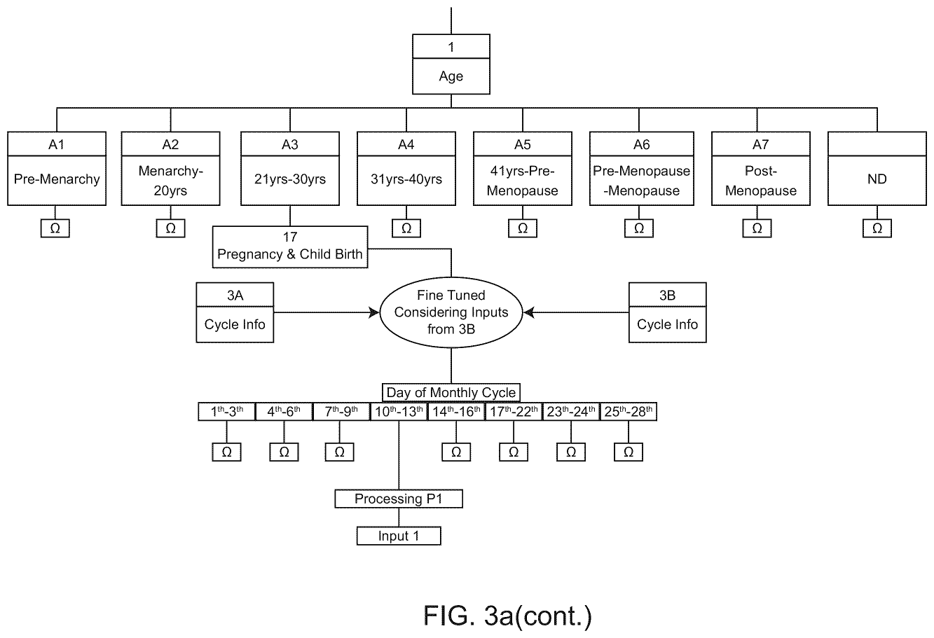

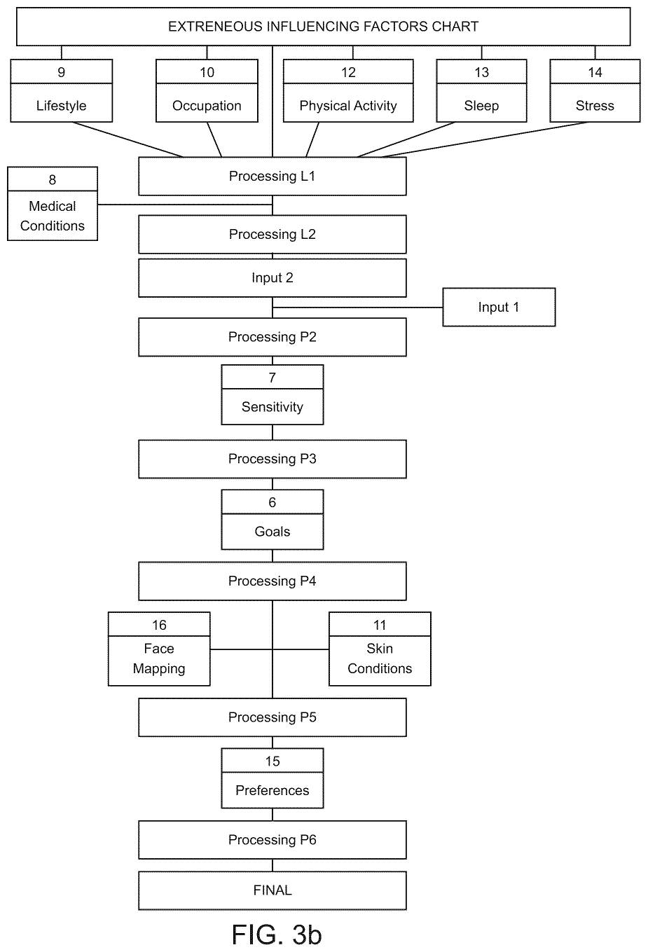

FIG. 3a illustrates an exemplary embodiment relating to the step-by-step process involved in the predictive, preventive, palliative, and curative care by utilizing the various information collected through the various means as explained in FIGS. 3a to 3b. The legend (2 in FIG. 3a corresponds to the other information as similar to exemplified flow. Initially, the process stars with assessing and defining the ethnicity (4), where the ethnicity classification comprising of E1 stands for Caucasian, E2 for Asian, E3 for Hispanic, E4 for African-American and ND for Not defined. The next processing step is analyzing the skin type of the user along with the ethnicity (4), where the input for skin type is obtained from the questionnaire (2A) and from the photo (2B). The confirmation of the skin type (2A) obtained through questionnaire and (2B) obtained through photo as illustrated in FIGS. 3a, and 3b provides output relating to the skin type such as oily skin, normal skin, dry skin, combinations skin or finer sub-categories of skin. If the skin type between (2A) and (2B) is not matched, then the tool considers skin type obtained through questionnaire (2A) to determine the output for skin type.

The output from the skin type processing step is further compared and correlated with the weather information obtained through questionnaire (5A) and from the smart devices (5B) and provides temperature details. In one embodiment, when the weather information between (5A) and (5B) is not matched, then the tool considers only the weather information obtained from the smart device (5B) to determine the output accordingly. The processing step correlates the output from the weather information parameter with the next temperature parameter classes and sub classes of relative humidity. Generally, the temperature parameter is classified as Temp <−10 C, Temp (−10) to (+10° C.), Temp (10) to (25° C.), Temp (25) to (30° C.), Temp (35) to (45° C.) and Temp >45° C. Each of these temperature parameter class further comprises subclass based on the relative humidity (RH). The subclass of the relative humidity is classified as <30, 30-50, 50-70, and >70, where each subclass of the relative humidity in conjunction with the temperature class indicates whether the particular skin type under these conditions will continue to behave as it is or will now change its behavior to another skin type . . .

Further, the processing step correlates the age parameter with the output of the previous step obtained from the weather and temperature processing step. The age parameter classification comprises but not limited to Pre-Menarche (A1), Menarche-20 yrs (A2), 21-30 years (A3), 31-40 years (A4), 40 years-Pre-menopause (A5), Pre-menopause-menopause (A6), post-menopause (A7) and ND for not defined. In one embodiment, the step additionally includes the pregnancy and childbirth parameter, wherein it will be considered as an additional factor. Furthermore, the output generated from the age, pregnancy and childbirth parameter is then correlated with the menstrual cycle information obtained from the questionnaire (3A) and the smart device (3B), which are compared with the day of the monthly cycle to provide a first input (input 1) using one or more modules of the processing step (P1).

FIG. 3b illustrates the extraneous influencing factors like lifestyle (9), occupation (10), physical activity (12), sleep (12) and stress (13) are compared and analyzed using the one or more modules of the processing step (L1). This extraneous information is correlated with the medical condition (8) parameter obtained from the questionnaire as illustrated in FIG. 2a, and further processed by one or more modules of the processing step (L2) to generate a second input (input 2).

The one or more modules of the processing step (P2) receives both the inputs from the first input and the second input and further processes the inputs using the one or more modules of the processing step (P3) by comparing and correlating with the sensitivity (7) obtained via the questionnaire as in FIG. (2a). Then the one or more modules of the processing step (P4) correlates the output from the sensitivity parameter with the goals (6) obtained from the questionnaire as in FIG. 2a. Then the processed information from (P4) is compared with face mapping (16) and skin conditions (11) obtained from the photos as in FIG. 2b and further processed by the one or more modules of the processing step (P5). At last, the output from the processing step (p5) is correlated with the user's preferences (15) obtained from the questionnaire as in FIG. 2a and processed by the one or more modules of the processing step (P6) to provide final predictive, preventive, palliative, and curative care-based suggestions, including the regimen to be followed, to the user by any one of the feedback means. This feedback is displayed on the screen as pop up, notification, alert, displayed as different icon or tab, a voice notes and/or combination thereof.

Another exemplary embodiment of the present disclosure comprising the step of obtaining parameters through a set of questionnaires, where the parameters are corresponding to age, skin type, cycle information, ethnicity, location, goals, sensitivity, medical information, lifestyle, occupation, and preferences. The skin type, skin condition and face mapping parameters are obtained through photos which is taken from camera of the user device. Further, the body temperature change, location, physical activity, stress, and sleep parameters are collected through the user's smart devices.

The detailed step-by-step process involved in the predictive, preventive, palliative, and curative care by utilizing the various parameters collected through the various means. The initial step is assessing and defining the user's ethnicity, where the ethnicity classification comprising Caucasian, Asian, Hispanic, African-American and others. The next step is analyzing the user's age in coordination with their ethnicity. The age parameter classification comprising of but not limited to Pre-Menarche, Menarche-20 yrs, 21-30 years, 31-40 years, 40 years-Pre-menopause, Pre-menopause-menopause, post-menopause and others. In this exemplary embodiment, the processing step initially analyzes the information such as ethnicity, age, and skin type information. For example, the skin type parameter obtained through photo and questionnaire are compared to identify the type of skin as oily, normal, dry and combination, or finer sub-categories and/or combinations thereof. In case the skin type is not matched with the other parameters then considering and analyzing only the skin type obtained through questionnaire which will be revalidated after several rounds of usage of this system and results thereof. In the next step the information is correlated with menstrual cycle information obtained through questionnaire. Further, all the correlated and confirmed information like ethnicity, age, skin type and cycle are processed by the one or more modules of the processing unit along with weather information obtained from questionnaire and smart devices/wearable devices to provide first input. The weather information is classified according to combinations of temperature bands and relative humidity groups.

In this exemplary method, the separate set of extraneous influencing factors like lifestyle, occupation, physical activity, sleep and stress are compared and analyzed using a processing unit. This extraneous information is correlated further with medical condition obtained from the questionnaire, and furthermore processed by processing unit to provide second input.

The one or more modules of the processing unit receives both the inputs from the first input and the second input and further correlates with sensitivity parameter from the questionnaire. All these information are processed using the processing unit followed by correlating with goals obtained from the questionnaire and further processed by the one or more modules of the processing unit. Then the processed information is correlated with additional parameters like face mapping and skin conditions obtained from camera/photo and additionally processed by the one or more modules of the processing unit. Finally, all the previously processed parameters are correlated with the user's preferences obtained from the questionnaire and processed by the one or more modules of the processing unit to provide final suggestion corresponding to a predictive, preventive, palliative, and curative care-based suggestions to the user by any one of the feedback means. The system of the present disclosure also provides regimen as feedback to the user. This feedback is displayed on the screen as pop up, notification, alert, displayed as different icon or tab, a voice notes and/or combination thereof.

The sequence of parameters (e.g., ethnicity, age, temperature, menstrual cycle, skin type, and others) processes is an example and on the same basis another sequence and set of correlation matrices can be formed to arrive at the same result. However, the present disclosure is not only limited to the disclosed sequence of parameters but also takes input in any order of sequence to provide preventive, palliative and curative care-based suggestions.

According to another exemplary embodiment, the system and method of the present disclosure discloses that the disclosure also provides preventive, palliative, curative recommendations to men. In case of men, the menstrual cycle monitoring device of the system will be in inactive state. In one embodiment, the menstrual cycle, and pregnancy and childbirth parameters are excluded. In another embodiment, the menstrual cycle parameter is excluded.

According to another embodiment of the disclosure, the system records and stores the parameters and its corresponding suggestions/indications/predictions in the memory. Based on the past data, the system determines the suggestions on similar parameters. In one example, on a hot summer day user's skin feels sensitive and irritable, which is prone to sun burn, so the system recommends a sun protect lotion with high SPF as alert/feedback.

The system and method of the present disclosure can be incorporated in a mobile as an application, a cosmetic base formulation vending machine, a computer, and other devices that are well known in the prior art.

According to one embodiment, this disclosure provides permissible information to the companies that research, develop or manufacture cosmetics, food, grocery items, personalized cosmetics, and other settings like hospitals, diet recommendation industry, promotional advertisement service, laser clinic, wellness centers, parlors, beauty device manufacturers, perfumeries, and the facial skin health care services. The present disclosure gives an opportunity to the end user to use personalized, specialty/functional cosmetics required respective to each person's age, weather, location, environment, lifestyle factors and other related factors.

In another embodiment there is an inbuilt software tool which includes the steps of receiving satisfaction information about the recommendations made through the correlation analysis multidimensional matrix.

In one embodiment, the user is female. In another embodiment, the user is a male.

Thus, the advantages of the present disclosure are, but not limited to a novel, system, and method for providing preventive, palliative, and curative suggestions for recommending a skin care cosmetic, a treatment suggestion, restorative skin care, preventive skin care, cosmetic skincare, haircare, mood/emotional upliftment and pain management and other aspects of well-being. The system and method of the present disclosure are considered as cost-effective and in-home based solutions as it reduces the consultation cost with an experienced dermatologists and provides suggestion daily, on a timely basis or whenever required.

Another advantage is to use technology to provide a methodology for providing a daily care recommendation and regimen based on the day of the monthly cycle and other parameters towards accentuated results which was hitherto not being done/could not be done by the dermatologists and other specialists.

The system explains to the user the grooming and wellness issues being addressed, suggests actives required to be used, recommends products containing the required actives with the requisite percentage, can take the user to a captive store, as also suggests the regime to be followed based on the monthly cycle and the other above given factors. The attempt is to minimize the number of products and depend upon the Regimen for daily alterations as far as possible.

Further, the present disclosure also recommends a solution prior to the encounters. For example, the system recommends an anti-ageing solution far before skin ageing commences. This is to delay the ageing process in advance. Moreover, the data generated is used for various other applications. For example, when the system predicts the day, the user would have hunger cravings, so that it recommends a healthy diet, or days that user is prone to buying the system would suggest goodies to the user, or days that the user is irritable, so the system suggests appropriate books/music/therapy etc.,

Although the disclosure has been described with regard to its embodiments, specific embodiments and various examples, which constitute the best mode presently known to the inventors, it should be understood that various changes and modifications as would be obvious to one having the ordinary skill in this art may be made without departing from the scope of the disclosure as set forth in the claims appended hereto.

1. A method for providing one or more suggestions to a user, the method being performed by a system comprising one or more user devices comprises one or more sensors, an input unit, an output unit and a transceiver, and the server comprising a database, a transceiver, and a processing unit having one or more modules, wherein the one or more user devices are communicatively coupled to the server via a communication network, the method comprising:
receiving, by the server, a request from the user via one or more user devices to provide the one or more suggestions;
extracting, by the server a corresponding one or more parameters of the user through the one or more user devices;
processing, by the server, a first set of parameters to generate a first input;
processing, by the server, a second set of parameters to generate a second input;
processing, by the server, a third set of parameters, the first input, and the second input to generate one or more suggestions; and
displaying, by the server using the output unit of the one or more user devices the one or more suggestions.

2. The method as claimed in claim 1, wherein the one or more suggestions are predictive, preventive, palliative and curative care.

3. The method as claimed in claim 1, the one or more sensors comprise an image sensor, a body temperature sensor, a location sensor, and a combination thereof.

4. The method as claimed in claim 1, wherein the one or more parameters comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy, and combinations thereof.

5. The method as claimed in claim 1, wherein the first set of parameters, the second set of parameters, and the third set of parameters are selected from the group comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy, and combinations thereof.

6. The method as claimed in claim 1, wherein the output unit is configured to deliver the one or more suggestions through a visual display, an audio-based suggestions, and a suggestive pop-up message on the one or more user devices.

7. A system for providing one or more suggestions to a user, the system comprising:
a. one or more user devices comprising one or more sensors, an input unit, an output unit and a transceiver, the one or more user devices are configured to collect one or more parameters of the user; and
b. a server comprising a database, a transceiver and a processing unit, the processing unit comprises one or more modules, the one or more modules are configured to process the one or more parameters for determining one or more suggestions,
wherein the determined one or more suggestions are transmitted to the output unit of the one or more user devices,
wherein the one or more suggestions are predictive, preventive, palliative and curative care,
wherein the server and the one or more user devices are configured to be communicatively coupled via a communication network.

8. The system as claimed in claim 7, wherein the one or more modules are configured to process a first set of parameters, a second set of parameters and a third set of parameters.

9. The system as claimed in claim 8, wherein the first set of parameters, the second set of parameters, and the third set of parameters are selected from the group comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy, and combinations thereof.

10. The system as claimed in claim 7, wherein the one or more parameters collected via the one or more user devices comprising of age, skin type, menstrual cycle information, body temperature change, ethnicity, location, goals, sensitivity, medical information/conditions, lifestyle, occupation, skin condition, physical activity, stress, sleep, preferences, face mapping, pregnancy, and combinations thereof.

11. The system as claimed in claim 7, wherein the one or more sensors comprise an image sensor, a body temperature sensor, a location sensor, and a combination thereof.

12. The system as claimed in claim 7, wherein the output unit for one or more user devices is configured to deliver one or more suggestions through a visual display, an audio-based suggestion, a suggestive pop-up message on user's mobile, personal computer, e-device screen from time to time or combinations thereof.
